(19) United States
(12) Patent Application Publication
(10) Pub. No.: US 2005/0137465 A1
(43) Pub. Date: Jun. 23, 2005

(54) System and method for remote monitoring in home activity of persons living independently

(75) Inventors: Paul Edward Cuddihy, Ballston Lake, NY (US); Meena Ganesh, Clifton Park, NY (US); Jenny Marie Weisenberg, Schenectady, NY (US); Catherine Mary Graichen, Malta, NY (US); Helena Goldfarb, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/747,554

(22) Filed: Dec. 23, 2003

(51) Int. Cl. A61B 5/00; A61B 10/00; G06F 17/00

(52) U.S. Cl. 600/300; 128/903; 128/904; 128/920

(57) ABSTRACT

A system and method for monitoring the activities of a resident living independently. Activity sensors distributed around the home collect data on activity in the home. The data is communicated to a remote monitoring center and analyzed. The data is buffered to ascertain whether an event or important transition has occurred in the home. The system also may be utilized to ascertain long-term mobility changes in the resident, deviations from normal sleep patterns, and deviations from a normal wake up time.

### BACKGROUND

The invention relates generally to a system and a method for monitoring the in-home activities of persons living independently, and more particularly to a system and a method for remotely monitoring the in-home activities of elderly or disabled persons who live independently.

With medical advancements and increased attention to proper nutrition and sufficient exercise, the populace in the western civilization is living longer. For example, the number of elderly persons residing in the United States is increasing, and with the advancing age of the baby boomer generation, the number of elderly persons in the United States will increase significantly over the next several decades. Additionally, increased awareness and understanding of various mental and physical disabilities has led to an increase in the number of persons having diminished mental and/or physical faculties living independently.

With the increase in elderly and disabled persons living independently has come anxiety that these elderly and disabled persons are safe and secure in their own residences. There is increased anxiety by the elderly and disabled living alone that they may become injured or incapacitated and be unable to summon assistance. That anxiety is often shared by loved ones living at a distance from the elderly and/or disabled living independently.

Currently, the anxiety felt by the elderly and disabled living alone, as well as the anxiety felt by their loved ones, is addressed through several avenues. One way to ease anxiety is through frequent visits to the home by a caregiver. Such visits can be intrusive, time consuming, and often inconvenient and not appreciated. Another way is for the elderly or disabled person to move out of the home and move into a facility better able to monitor his health. This, however, strips the person of his independence, is costly and is often unwelcome. Another way is through technological assistance or monitoring of the person in the home.

Such technological systems that assist persons in their home include Personal Emergency Response Systems. In these systems the elderly or disabled individual wears a watch, pendant or other like device and presses a button in the event of an emergency, such as a fall. The depressed button enables an alarm signal. A central monitoring facility provides assistance by responding to the alarm signal and calls the individual to identify the problem. The facility calls a predetermined list of contacts, such as relatives, neighbors or emergency services, as required by the context of the situation. While a valuable service, these systems only identify problems that occur when the individual is able to press the emergency button.

Some known in-home monitoring systems collect data obtained from sensors and send the sensor data out of the home to a remote monitoring site using the phone system of the home. These phone calls are placed at set intervals. If the intervals are placed close together, there is often increased cost due to the increased use of the phone. Further, the frequent use of the phone is sometimes inconvenient, since the phone line is tied up at fixed, frequent intervals. The inconvenience is felt both by the resident of the home and by those attempting to contact the resident.

Thus, there remains a need, which is increasing, for an economical in-home monitoring system which limits its intrusiveness while providing caregivers a realistic view of the activities of the person residing in the home.

### BRIEF DESCRIPTION OF THE DRAWINGS

FIG. 1 is a schematic view of a remote in-home monitoring system in accordance with an embodiment of the invention.

FIG. 2 is a process for remote in-home monitoring by the system of FIG. 1.

FIG. 3 illustrates exemplary steps for communicating data to a remote monitoring center in the process of FIG. 2.

FIG. 4 illustrates steps for ascertaining the mobility of a resident of a home equipped with the system of FIG. 1.

FIG. 5 illustrates a method for ascertaining sleep patterns of a resident of a home equipped with the system of FIG. 1.

FIG. 6 illustrates a hidden Markov modeling technique for analyzing sleep patterns in accordance with the method of FIG. 5.

FIG. 7 illustrates steps for ascertaining late wake-up of a resident of a home equipped with the system of FIG. 1.

### SUMMARY

The present invention describes a system and a method for remote monitoring of a person through the use of sensors placed in the home and a processing system at a remote location for collating data obtained from the sensors and for contacting caregivers if warranted.

One aspect of the invention is an activity monitoring system for allowing a caregiver to monitor activity of a resident residing independently in a home. The system includes at least one activity sensor positioned within the home for collecting data on activity in the home, a near real-time communication platform in communication with the at least one activity sensor, and a monitoring center located remote from the home and in communication with the near real-time communication platform, wherein the near real-time communication platform is adapted to communicate data from the at least one activity sensor to the monitoring center in near real-time to the occurrence of an event.

Another aspect of the invention is an activity monitoring system for allowing a caregiver to remotely monitor activity of a resident residing independently in a home. The system includes a plurality of activity sensors positioned within the home for collecting data on activity in the home, a near real-time communication platform in communication with the plurality of activity sensors, and a monitoring center located remote from the home and in communication with the near real-time communication platform. The near real-time communication platform comprises one or more communication media in the group consisting of wired telephone, wireless telephone, two-way walkie-talkie, pager, cable, and the Internet. Further, the near real-time communication platform is adapted to communicate data from the plurality of activity sensors to the monitoring center in near real-time to the occurrence of an event.

Another aspect of the invention is a method for allowing a caregiver to monitor activity of a resident residing independently in a home. The method includes the steps of providing activity sensors for distribution throughout the home, collecting data from the activity sensors, communicating, via a near real-time communication platform, the data collected from the activity sensors to a monitoring center remote from the home in near real-time to the occurrence of an event, analyzing the data at the monitoring center, and generating a report to the caregiver upon the occurrence of the event.

Another aspect of the invention is a method for allowing a caregiver to remotely monitor activity of a resident residing independently in a home. The method includes the steps of providing activity sensors for distribution throughout the home, collecting data from the activity sensors, and communicating, via a near real-time communication platform, the data collected from the activity sensors to a monitoring center remote from the home in near real-time to the occurrence of an event. The method further includes analyzing the data at the monitoring center and generating an event report to the caregiver upon the occurrence of the event. The method also includes the step of generating, upon request of the caregiver, a status report of activity within the home.

Another aspect of the invention is a method for allowing a caregiver to monitor the mobility of a resident residing independently in a home. The method includes the steps of providing activity sensors for distribution throughout the home, collecting data from the activity sensors, communicating, via a near real-time communication platform, the data collected from the activity sensors to a monitoring center remote from the home, searching for activity patterns in the data collected from the activity sensors, studying the amount of time required to accomplish particular activities, and reporting to the caregiver long-term changes in the amount of time required to accomplish the particular activities.

Another aspect of the invention is a method for allowing a caregiver to monitor the sleep patterns of a resident residing independently in a home. The method includes the steps of providing activity sensors for distribution throughout the home, collecting data from the activity sensors, communicating the data collected from the activity sensors to a database via a near real-time communication platform, and analyzing the data collected from the activity sensors using a hidden Markov modeling technique to determine if the data indicates a deviation from the comparison data, signaling an abnormal sleep pattern.

Another aspect of the invention is a method for allowing a caregiver to monitor the wake up times of a resident residing independently in a home. The method includes the steps of providing activity sensors for distribution throughout the home, collecting data from the activity sensors indicating a wake up time of the resident, communicating the collected data to a monitoring center remote from the home via a near real-time communication platform, and analyzing the data collected from the activity sensors to determine if the data indicates that the resident is not awake by the predetermined normal wake up time, signaling an abnormal wake up time.

These and other advantages and features will be more readily understood from the following detailed description of preferred embodiments of the invention that is provided in connection with the accompanying drawings.

### DETAILED DESCRIPTION OF PREFERRED EMBODIMENTS

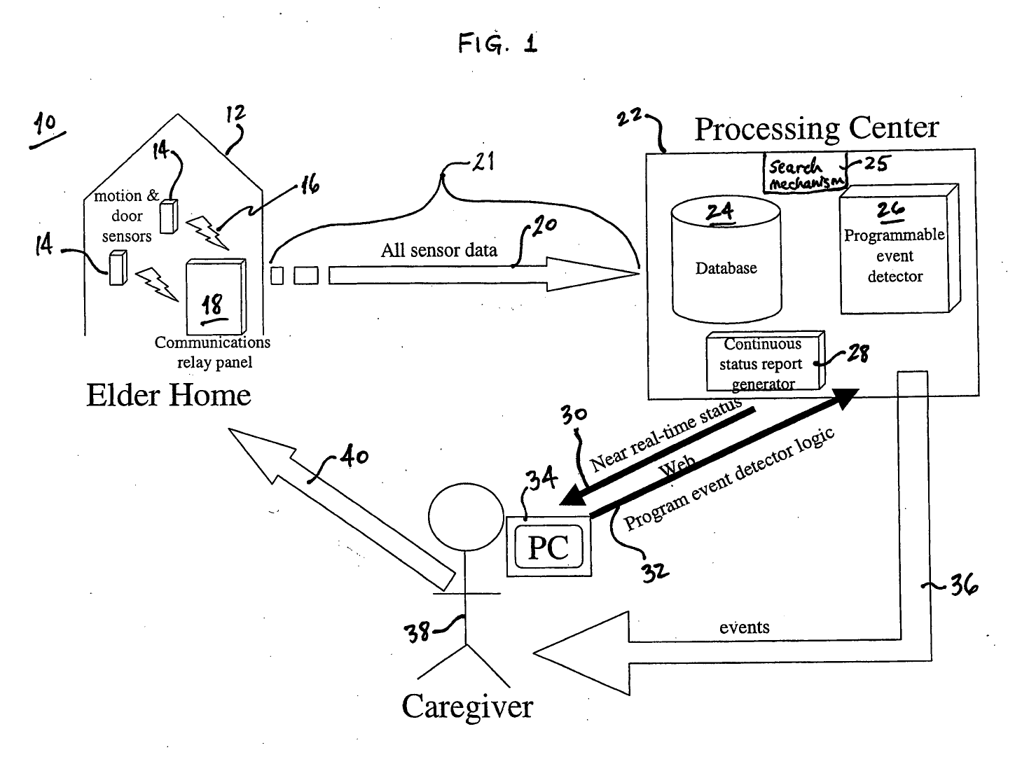

With reference to FIG. 1, there is shown an activity monitoring system 10 that includes activity sensors 14, a communication relay panel 18, and a remote monitoring center 22. The activity monitoring system 10 lacks mechanisms to intervene in the home 12 or any subsystems (appliances, water, lights, etc.) of the home 12. Intervention in the home 12, if any, may arrive through a communication with the resident of the home 12 from outside the home, such as via a telephone call or a visit from a caregiver 38 or other suitable person, such as an emergency response professional. The sensors 14 include motion sensors, door sensors, and any other sensors suitable for collecting and communicating data regarding activities on-going in the home 12. Other suitable sensors 14 include hazard sensors and security sensors. Preferably, the sensors 14 are wireless sensors capable of wirelessly communicating signals 16, which include data collected, to the communications relay panel 18. It should be appreciated, however, that the sensors 14 instead may be sensors wired to the communications relay panel 18.

The communications relay panel 18 communicates the sensor data collected from the sensors 14 by sending a data signal 20 to the remote monitoring center 22 by way of a suitable wired or wireless communications platform 21, such as, for example, wired telephone, wireless telephone, two-way walkie-talkie, pager, cable, the Internet browser, or any other wireless communication platform. Depending upon the communication platform 21 chosen, the data signals 20 may be sent in near real-time or may be sent at discrete, irregular intervals. For example, data signals 20 may be sent in near real-time via wireless telephone, two-way walkie-talkie, pager, cable, the Internet browser or any other wireless communication platform. For a wired telephone communication platform, the data signals 20 are buffered and transmitted at differing intervals.

The monitoring center 22, which is remote from the home 12, includes a database 24, a programmable event detector 26, and a continuous status report generator 28. The database 24 serves as a collection vessel for the sensor data communicated via the signals 20. Upon a request from the caregiver 38 for a status report, the sensor data is forwarded from the database 24 to the continuous status report generator 28. The status report generator 28 communicates a near real-time status signal 30 to a personal computer 34 of the caregiver 38. By near real-time is meant anywhere in the range of almost instantaneously to up to three minutes. For example, for a two-way page communication platform 21, the amount of time required for the communication can be between two and three minutes. The status report generator 28 may be programmed to update the report for each home 12 at a certain interval, such as, for example, every ten minutes. The status signal 30 includes a report generated by the continuous status report generator 28. The format and substance of the report are dependent upon the request of the caregiver 38. It should be appreciated that the signal 30 can instead be communicated via a personal digital assistant (PDA), a pager, a facsimile machine, cable, or a telephone or voice-mail account instead of via the personal computer 34.

The caregiver 38 can also select certain activities that, if they occur in the home 12, would be considered an event. An event, in general, would include an activity or any important transition occurrence, such as a state transition (the change from one state to another, such as, for example, from active to quiet), of which a caregiver 38 would want to be apprised. For example, the time of wake up, an unusually long period of quiet or no activity, or the use of an exterior door may be considered an important activity or state transition occurrence. The caregiver 38 communicates the parameters of what constitutes an event to the remote monitoring center 22 via a signal 32. While the caregiver 38 does not define what constitutes an event, the caregiver 38 can select which from a set of predefined activities constitutes an event. Further, the caregiver 38 sets the parameters to configure the events to match the normal activity of the resident in the home 12. For example, the caregiver 38 does not define what constitutes, for example, “wake up”, but the caregiver 38 can define when “wake up” would be considered late. The sensor data is stored and processed at the monitoring center 22. If the data indicates the occurrence of an event, a signal 36 is sent to the caregiver 38 via any suitable communication medium, such as, for example, wired or wireless telephone, PDA, pager, facsimile, cable, two-way walkie-talkie, e-mail, or other Internet-supported communication media, such as, for example, through a pop-up announcement format. The caregiver 38 is then provided the opportunity to open a communication pathway 40 with the person residing in the home 12. The communication pathway 40 may be through a wired or wireless telephone line, the Internet browser (i.e., e-mail or other Internet-sponsored communication tool), cable, PDA, pager, or personal, such as a visit by the caregiver 38 or another suitable person.

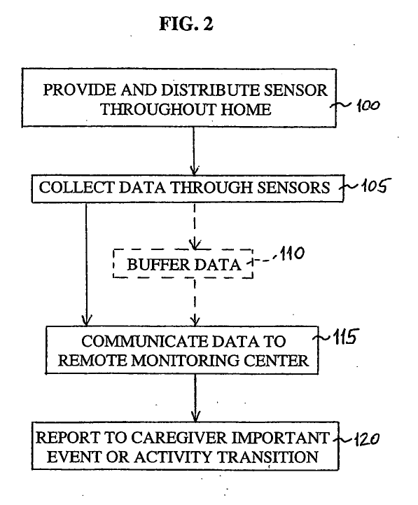

Next, with specific reference to FIG. 2, will be described a method of remote monitoring of a person within the home 12. At Step 100, sensors 14 are provided and distributed throughout the home 12. The arrangement of the sensors 14 throughout the home 12 depends upon the configuration of the house and the areas where activity/motion are more likely to occur and in which the collected data provides a more meaningful accounting of activity. Further, the arrangement of the sensors 14 may be impacted by the cost of the sensors 14 as well as the issue of privacy.

Next, at Step 105, data is collected from the sensors 14. The collection of the data may be accomplished through the wireless communication of the data from the activity sensors 14 to the communications relay panel 18 via the data signal 20. The communications relay panel 18 collects the data and holds it until communicated to the remote monitoring center 22 at Step 115. The amount of time the data is held by the communications relay panel 18 depends upon the communication platform 21. If the communication platform 21 between the communications relay panel 18 and the remote monitoring center 22 is wireless (wireless telephone, two-way pager, PDA, Internet browser, cable, etc.), the transmission of the data signals 20 is in near real-time. It should be appreciated that a communications relay panel 18 may not be necessary with a wireless communication platform 21, and the sensors 14 may instead be in direct communication with the remote monitoring center 22 through the wireless communication platform 21.

If, on the other hand, the communication platform 21 is a wired telephone, the data signals 20 are first buffered at optional Step 110. The buffering Step 110 is for culling through the data signals 20 to ascertain whether any of the signals 20 indicates an important transition between activity and inactivity for either the entire system or a subset of the system. Upon such indication, the data signals 20 (or some subset of them) are then communicated to the remote monitoring center 22. As noted above, the communication of the data to the remote monitoring center 22 may be through any suitable communication platform 21, such as, for example, wired telephone, wireless telephone, two-way walkie-talkie, pager, cable, PDA, or the Internet browser. The data is collected at the database 24 of the remote monitoring center 22 and is kept in a way that allows for quick preparation of status reports by the status report generator 28 upon request by the caregiver 38.

After the communication Step 115, at Step 120 a report is provided to the caregiver 38 reporting an event. As noted above, the event is selected by the caregiver 38, which ensures that its occurrence is of high concern to the caregiver 38. The report is generated by the event detector 26 and communicated to the caregiver 38 through any suitable communication medium, such as, for example, wired or wireless telephone, pager, two way walkie-talkie, facsimile, cable, e-mail, or other Internet-supported communication media, such as through a pop-up announcement format.

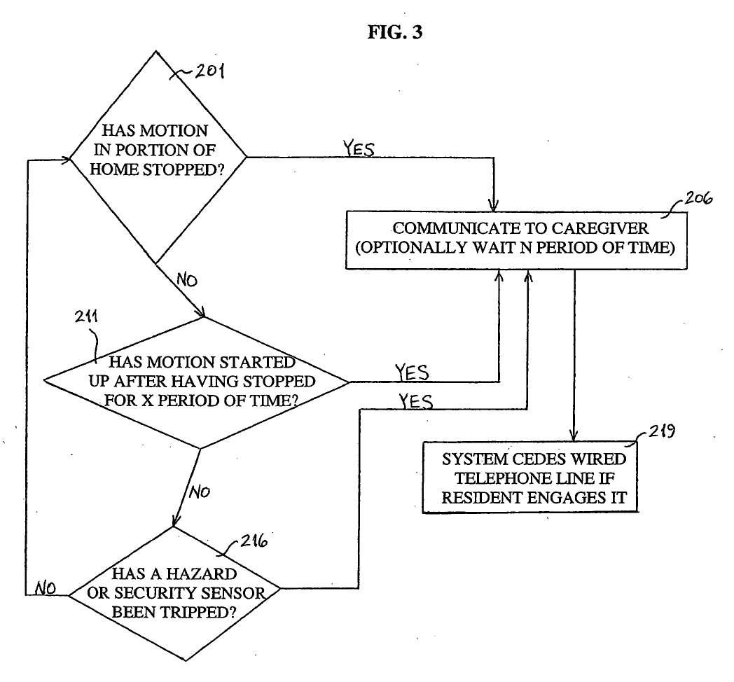

With specific reference to FIG. 3, next will be described a method for communicating to the caregiver 38 via wired telephone in near real-time to the occurrence of an event. As noted above, current in-home monitoring systems generally report out data collected from sensors to a remote processing center at a fixed interval over a phone line. One disadvantage of this is the inconvenience, to the home resident and to those calling in, of the phone line being in use. Another disadvantage is that data that is collected may not be reported for a substantial period of time, such as an hour or longer. As noted above, utilizing a communication platform 21 other than wired telephony, such as wireless telephone, cable, two-way walkie-talkie, pager, or the Internet browser obviates one disadvantage to communicating via wired telephony. By utilizing a communication platform 21 not linked to the wired telephony infrastructure, data can be more freely transmitted to the remote monitoring center 22 at a lower cost. Thus, more frequent communication of the data signal 20 may occur, thereby minimizing the amount of time between the acquisition of data from the sensors 14 and the communication of the data signals 20.

Nonetheless, FIG. 3 provides a methodology for utilizing a wired telephony communication platform 21 for communicating data signals 20 in near real-time to the occurrence of an event. This methodology utilizes a buffering technique (Step 110 in FIG. 2) to cull through the totality of the data stored in the communications relay panel 18 to ascertain whether any of that data indicates the occurrence of an event, such as a state transition. At logic node 201, an inquiry is made in the communications relay panel 18 whether the data currently residing therein indicates whether important motion in a particular area within the home 12 has stopped. Important motion would include such motion that, if one knew it had stopped, would cause an alarm. If the answer to that inquiry is yes (meaning a state transition has occurred), at Step 206 the communications relay panel 18 uses the wired telephony communication platform 21 to transmit at least that data evidencing the state transition (if not all or some larger subset of the data stored in the communications relay panel 18) to the remote monitoring center 22. The programmable event detector 26 reports the event, namely the ceasing of activity in that particular area, to the caregiver 38. Optionally, the event detector 26 can wait an N period of time to allow the collection and communication of more data to ascertain whether activity in that particular area has resumed. The period of time for waiting N may be any suitable time, such as, for example, fifteen minutes. Further, the period of time for waiting can be tailored to the portion of the home experiencing the movement just prior to movement ceasing. At Step 219 the monitoring system 10 cedes the wired telephone line if the resident engages the telephone line.

If instead the answer to the inquiry is no, then at logic node 211, an inquiry is made whether the data collected at the communications relay panel 18 indicates that important motion has started up after having stopped for an X period of time. If the answer to the inquiry is yes (state transition), then the data indicating the state transition (or some larger subset of data) is communicated to the remote monitoring center 22 and a report is generated by the event detector 26 and reported to the caregiver 38 at Step 206. If the answer to the inquiry is no, then a further inquiry is made.

At logic node 216, an inquiry is made whether the data indicates that a hazard or security sensor, such as, for example, a temperature, fire, or smoke alarm or a panic button, has been tripped in the home 12. If the answer is yes (state transition), again the data indicating the state transition (or some larger subset of data) is communicated to the remote monitoring center 22 and a report is generated by the event detector 26 and reported to the caregiver 38 at Step 206. If the answer is no, then the communications relay panel 18 returns to the logic node 201 and awaits the latest update of data to recommence the sequence of inquiries.

It should be appreciated that the inquiries in the logic nodes 201, 211, and 216 are exemplary and are not intended to be exhaustive of the type of inquiries that can be made, nor are they exhaustive of the ordering/structure of the series of logic nodes. Alternatively, there may be multiple checks and interdependencies among the logic nodes 201, 211, 216. For example, an inquiry can be made as to whether activity has started in a particular location, such as a bathroom or a kitchen, and has stopped within a pre-determined period of time. One possible inquiry could be whether water is detected filling a bathtub, and the length of time the water has been running. Another possible inquiry could be whether a stove has been engaged and for how long it has been running. It should also be appreciated that the period of time for waiting before generating an event report may be tailored depending upon the location of the activity sensor 14 collecting data on the activity. For example, sensors in the kitchen may be set for a longer waiting period of time, such as twelve hours, before a report is generated.

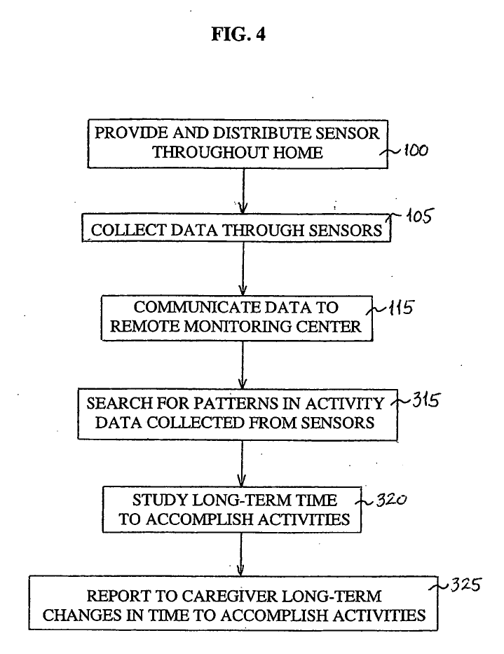

One particular concern of caregivers 38 is the long-term change in a resident's mobility. Speed of movement is a good predictor of changes in health. Oftentimes, long-term changes in activity are difficult to quantify or verify through continuous observation. As will be described next with specific reference to FIG. 4, the long-term mobility of a resident of a home 12 can be quantified through the system 10.

At Step 100, activity sensors 14 are provided and distributed through the home 12. The sensors 14 are used to collect data at Step 105. The data so collected is communicated to the database 24 at the remote monitoring center 22 at Step 110. Then, at Step 315, the data stored in the database 24 is searched by a search mechanism 25 (FIG. 1) for patterns in activity. The more useful patterns are those that occur several times a week, such as, for example, data collected from the sensors 14 that indicates activity in the kitchen at mealtime or activity in the bedroom at or near bedtime. Pattern matching algorithms are used to find common patterns that occur on most days.

Then, at Step 320, the completion time for each activity is inferred and studied. The studying may be accomplished through any number of known algorithmic methods. For example, the longest period of time to finish an activity may be mapped over a predetermined time period, such as two months, to develop the change in time over that time period it takes the resident to perform that activity. Alternatively, all the periods of time necessary to finish an activity may be mapped over a predetermined time period. The outlier data, the data existing at the boundaries (such as, for example, 25 percent of the data representing the longest and shortest time periods) may be thrown out and the remaining data used to develop the change in time over that time period it takes the resident to perform that activity.

Finally, at Step 325, the caregiver 38 receives a report on the long-term changes in time it takes the resident to accomplish certain inferred and studied activities. Providing caregivers 38 a report at an earlier timeframe indicating a quantifiable change in mobility of the resident of the home 12 may lead to increased awareness of a change in condition. For example, a change in mobility may be traced to a change in medication, or it may be traced to a change in mood of the resident. Earlier detection of a change in mobility can lead to earlier diagnosis of the cause behind the change in mobility, and hence, can lead to earlier treatment of the conditions causing the change in mobility. It should be appreciated that multiple patterns may be concurrently studied in the home 12, thereby ensuring robustness for the studying. For example, if long-term changes seem to indicate an increase in the amount of time to accomplish one task and a decrease in the amount of time needed for another task, there may be factors at work other than mobility. On the other hand, if data seems to indicate long-term increases in time to accomplish numerous tasks, then that seems to confirm that the resident's mobility has decreased.

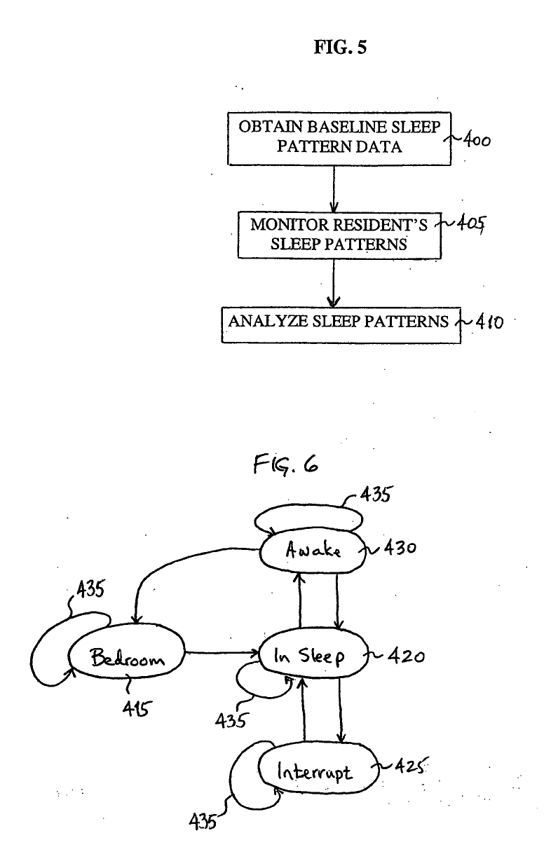

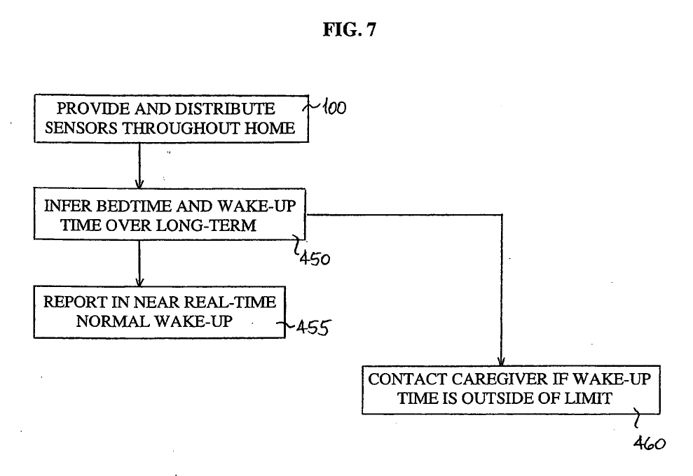

Changes in sleep patterns, like changes in mobility patterns, can signal a medical problem. For example, a change in sleep patterns may be an indicator of depression, or it may be an indicator that a medication needs to be changed or that a recent change in medication is affecting the resident's sleep patterns. FIG. 5 illustrates an exemplary method for chronicling the sleep patterns of a resident of the home 12 to ascertain whether any particular sleep pattern is normal or abnormal. FIG. 6 illustrates one exemplary embodiment for analyzing sleep patterns utilizing a hidden Markov modeling technique.

As shown in FIG. 5, the method begins with a training period at Step 400. The training period Step 400 is used to obtain baseline sleep pattern data on the resident of the home 12. The length of the training period should be sufficient to provide baseline sleep pattern data for all the resident's sleep patterns. Sleep patterns are determined by the amount of sleep, the location of the sleep, and the frequency and duration of any interruptions to sleep. These factors, as well as the day of the week (weekend versus weekday, for example) often lead to multiple sleep patterns per resident. The length of the training period may be as short as seven days or as long as one month or longer.

Once the baseline sleep pattern data has been obtained, data is collected on the resident in the home 12 to facilitate monitoring of the resident's sleep patterns at Step 405. Finally, at Step 410, the sleep pattern data is analyzed to ascertain whether the data supports a conclusion that the resident's most recent sleep pattern is normal or abnormal with reference to the baseline sleep pattern data. If the data supports a conclusion that the most recent sleep pattern is abnormal, a report may be generated and communicated to the caregiver 38 via communication media described above. Alternatively, a further analysis step may be performed to ascertain whether the abnormality of the most recent sleep pattern is sufficiently abnormal to warrant a report to the caregiver 38. Whether a sleep pattern is considered sufficiently abnormal may be determined by a predetermined set of rules, feedback from the caregiver (which may assist in retraining the home), or a combination of the two.

One exemplary method for analyzing sleep pattern data is through a hidden Markov modeling technique, which is described with reference to FIG. 6. A Markovian property is that given the present state, the next state to occur is independent of all previous states. This leads to the inference that the transition between specific states is probabilistic, and therefore can be modeled. A hidden Markov modeling technique is a technique for modeling a symbolic sequence. It is a probabilistic pattern matching approach that models a sequence of patterns as the output of a random process.

As illustrated in FIG. 6, there are four states possible that relate to a resident's sleep patterns, namely the bedroom 415, in sleep 420, interrupted sleep 425, and awake 430. The transitions between the states 415, 420, 425, 430 are noted, and these state transitions are identified and compared to the baseline sleep pattern data obtained at Step 400. The in sleep state 420 is a quiet period during a period of time in which sleep would be expected. The term “quiet” is a period where no activity happens in the home 12 for more than 15 minutes. The location before the in sleep state 420 is inferred is marked as the bedroom state 415. Any activity that happens after the in sleep state 420 is marked as either an interrupt state 425 or an awake state 430. The difference between the interrupt and awake states 425, 430 is that the awake state is an interrupt state that has lasted for more than 30 minutes.

With reference to FIGS. 5, 6, data is taken from the activity sensors 14 for a training period of time to set up a database of baseline sleep pattern data at Step 400. The sleep pattern data may automatically separate into similar sleep patterns. After sufficient baseline sleep pattern data has been obtained, various patterns of sleep will have been identified as being normal for a resident. Then, at Step 405, the resident's sleep patterns are monitored. A hidden Markov modeling technique is utilized to analyze the sleep patterns at Step 410. Transitions between the states are done in ten minute slices 435. This methodology accounts for a number of transition slices. Thus, if a resident's in sleep state 420 lasts for four hours, then the count of transition from in sleep state to in sleep state will be a count of twenty-four. The baseline sleep pattern data is used to predict whether the resident's most recent sleep pattern belongs to a previously identified sleep pattern, and therefore is normal, or whether it does not belong to any previously identified sleep pattern, and therefore is abnormal for that resident. If the resident's sleep pattern is determined to be abnormal, a report is generated identifying a deviation in the resident's sleep pattern.

Another indicator that there may be a problem with a resident of the home 12 is a failure to wake up at a time normal to the resident. Caregivers 38 often worry that a resident may be ill or incapacitated in bed, unable to contact anyone for help, and remaining incapacitated for a lengthy period of time before anyone realizes the problem. As illustrated in FIG. 6, the system 10 may be used to model wake-up behavior and report to the caregiver 38 when any wake-up time is outside the acceptable limits.

At Step 100, the sensors 14 are provided to and distributed throughout the home 12. For a period of time, the bedtime and wake-up times of the resident are recorded at Step 450. By inferring bedtime and wake-up times over a period of time, more accurate data can be compiled pertaining to the normal length of the sleeping period of the resident. This data is used to ascertain sleep patterns. One approach is to utilize the hidden Markov model technique described above with reference to FIG. 5. An alternative approach is to look for lengthy periods of quiet in the home 12, such as six to eight hours. Alternatively, the data can be searched to ascertain the latest wake-up time for a period of time, such as the last 60 days, and a report can be generated if the wake-up time exceeds that period of time by a certain amount, such as by one hour.

When the resident wakes up at a normal time, meaning within the predetermined limits based upon the analyzed historical sleep pattern data, at Step 455 an undated status report is generated and sent to the caregiver 38 in near real-time reporting the normal wake-up time. If, on the other hand, there is no wake up identified by the end of the predetermined limit, the caregiver 38 may be contacted at Step 460. Any suitable method for contacting the caregiver 38 may be used, such as, for example, wired or wireless telephone, pager, two way walkie-talkie, facsimile, cable, e-mail, or other Internet-supported communication media, such as through a pop-up announcement format.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. An activity monitoring system for allowing a caregiver to monitor activity of a resident residing independently in a home, comprising:
at least one activity sensor positioned within the home for collecting data on activity in the home;
a communication platform in communication with the at least one activity sensor; and
a monitoring center located remote from the home and in communication with the communication platform;
wherein the communication platform is adapted to communicate the data from the at least one activity sensor to the monitoring center and the monitoring center is adapted to analyze the data.

2. The system of claim 1, further comprising a communications relay panel positioned within the home and in communication with the at least one activity sensor and the communication platform.

3. The system of claim 2, wherein the communication platform comprises one or more communication media in the group consisting of wired telephone, wireless telephone, two-way walkie-talkie, pager, cable, and the Internet browser.

4. The system of claim 2, wherein the monitoring center comprises:
a database for receiving and compiling the data on activities in the home from the communications relay panel; and
an event detector for communicating an event report to the caregiver in near real-time to an occurrence of an event.

5. The system of claim 1, wherein the monitoring center is adapted to communicate with the caregiver through one or more communication media in the group consisting of wired telephone, wireless telephone, pager, two way walkie-talkie, facsimile, cable, e-mail, and pop-up Internet-supported announcement.

6. The system of claim 1, further comprising a status report generator for generating a near real-time status report upon request by the caregiver.

7. The system of claim 1, wherein the at least one activity sensor comprises a plurality of wireless sensors.

8. The system of claim 7, wherein the plurality of wireless sensors comprises an activity sensor for detecting activity in a kitchen.

9. The system of claim 7, wherein the plurality of wireless sensors comprises an activity sensor for detecting activity at a doorway providing ingress and egress to the home.

10. The system of claim 7, wherein the plurality of wireless sensors comprises an activity sensor for detecting activity in a bedroom.

11. The system of claim l, wherein the monitoring center comprises a search mechanism adapted to search for patterns in activity of the resident.

12. The system of claim 1, wherein the monitoring center is adapted to ascertain whether the resident experiences a deviation in his sleep pattern.

13. The system of claim 1, wherein the monitoring center is adapted to ascertain whether the resident experiences a deviation from his normal wake up time.

14. The system of claim 17 wherein the caregiver determines which criteria from a predefined set of criteria constitutes an event.

15. An activity monitoring system for allowing a caregiver to remotely monitor activity of a resident residing independently in a home, comprising:
a plurality of activity sensors positioned within the home for collecting data on activity in the home;
a near real-time communication platform in communication with the plurality of activity sensors wherein the near real-time communication platform comprises one or more communication media in the group consisting of wired telephone, wireless telephone, two-way walkie-talkie, pager, cable, and the Internet browser, and
a monitoring center located remote from the home and in communication with the near real-time communication platform;
wherein the near real-time communication platform is adapted to communicate the data from the plurality of activity sensors to the monitoring center and the monitoring center is adapted to analyze the

16. The system of claim 15, further comprising a communications relay panel positioned within the home and in communication with the plurality of activity sensors and the near real-time communication platform.

17. The system of claim 15, wherein the monitoring center is adapted to communicate with the caregiver through one or more communication media in the group consisting of wired telephone, wireless telephone, pager, two way walkie-talkie, facsimile, cable, e-mail, and pop-up Internet-supported announcement.

18. The system of claim 15, further comprising a status report generator for generating a near real-time status report upon request by the caregiver.

19. The system of claim 15, wherein the plurality of wireless sensors comprises an activity sensor for detecting activity in a kitchen.

20. The system of claim 15, wherein the plurality of wireless sensors comprises an activity sensor for detecting activity at a doorway providing ingress and egress to the home.

21. The system of claim 15, wherein the plurality of wireless sensors comprises an activity sensor for detecting activity in a bedroom.

22. The system of claim 15, wherein the monitoring center comprises a search mechanism adapted to search for patterns in activity of the resident.

23. The system of claim 15, wherein the monitoring center is adapted to ascertain whether the resident experiences a deviation in his sleep pattern.

24. The system of claim 15, wherein the monitoring center is adapted to ascertain whether the resident experiences a deviation from his normal wake up time.

25. The system of claim 15, wherein the caregiver determines which criteria from a predefined set of criteria constitutes an event.

26. A method for allowing a caregiver to monitor activity of a resident residing independently in a home, comprising the steps of:
providing activity sensors for distribution throughout the home;
collecting data from the activity sensors;
communicating, via a communication platform, the data collected from the activity sensors to a monitoring center remote from the home in response to an occurrence of an event;
analyzing the data at the monitoring center; and
generating an event report to the caregiver upon the occurrence of the event.

27. The method of claim 26, wherein the step of collecting data comprises wirelessly communicating the data obtained by the activity sensors to a communications relay panel.

28. The method of claim 27, wherein the step of communicating the data comprises communicating the data from the communications relay panel to the monitoring center via the communication platform.

29. The method of claim 28, wherein the communication platform comprises one or more communication media from the group consisting of wired telephone, wireless telephone, two-way walkie-talkie, pager, cable, and the Internet browser.

30. The method of claim 26, further comprising communicating the event report to the caregiver.

31. The method of claim 30, wherein the step of communicating the event report comprises communicating the event report via one or more communication media from the group consisting of wired telephone, wireless telephone, pager, two way walkie-talkie, facsimile, cable, e-mail, and pop-up Internet-supported announcement.

32. The method of claim 26, further comprising generating, upon request of the caregiver, a status report of activity within the home.

33. The method of claim 32, further comprising communicating the status report to the caregiver via one or more communication media from the group consisting of a pager, a facsimile machine, cable, a wired or wireless telephone or voice-mail account, and a personal computer.

34. A method for allowing a caregiver to remotely monitor activity of a resident residing independently in a home, comprising the steps of:
providing activity sensors for distribution throughout the home;
collecting data from the activity sensors;
communicating, via a near real-time communication platform, the data collected from the activity sensors to a monitoring center remote from the home in near real-time to the occurrence of an event;
analyzing the data at the monitoring center;
generating an event report to the caregiver upon the occurrence of the event;
communicating the event report to the caregiver; and
generating, upon request of the caregiver, a status report of activity within the home.

35. The method of claim 34, wherein the step of collecting data comprises wirelessly communicating the data obtained by the activity sensors to a communications relay panel.

36. The method of claim 35, wherein the step of communicating the data comprises communicating the data from the communications relay panel to the monitoring center via the near real-time communication platform.

37. The method of claim 36, wherein the near real-time communication platform comprises one or more communication media from the group consisting of wired telephone, wireless telephone, two-way walkie-talkie, pager, cable, and the Internet browser.

38. The method of claim 34, wherein the step of communicating the event report comprises communicating the event report via one or more communication media from the group consisting of wired telephone, wireless telephone, pager, two way walkie-talkie, facsimile, cable, e-mail, pop-up Internet-supported announcement.

39. The method of claim 34, further comprising communicating the status report to the caregiver via one or more communication media from the group consisting of a pager, a facsimile machine, cable, a wired or wireless telephone or voice-mail account, and a personal computer.

40. A method for allowing a caregiver to monitor the mobility of a resident residing independently in a home, comprising the steps of:
providing activity sensors for distribution throughout the home;
collecting data from the activity sensors;
communicating, via a communication platform the data collected from the activity sensors to a monitoring center remote from the home;
searching for activity patterns in the data collected from the activity sensors;
studying the amount of time required to accomplish particular activities; and
reporting to the caregiver long-term changes in the amount of time required to accomplish the particular activities.

41. The method of claim 40, wherein the step of collecting data comprises wirelessly communicating the data obtained by the activity sensors to a communications relay panel.

42. The method of claim 41, wherein the step of communicating the data collected from the activity sensors comprises communicating the data to the monitoring center via one or more communication media from the group consisting of wired telephone, wireless telephone, two-way walkie-talkie, pager, cable, and the Internet browser.

43. A method for allowing a caregiver to monitor the sleep patterns of a resident residing independently in a home, comprising the steps of:
providing activity sensors for distribution throughout the home;
collecting data from the activity sensors;
communicating the data collected from the activity sensors to a database via a communication platform; and
analyzing the data collected from the activity sensors using a hidden Makov modeling technique to determine if the data indicates a deviation from the comparison data, signaling an abnormal sleep pattern.

44. The method of claim 43, further comprising generating a report to the caregiver if the data indicates a deviation from the comparison data, signaling an abnormal sleep pattern.

45. A method for allowing a caregiver to monitor the wake up times of a resident residing independently in a home, comprising the steps of:
providing activity sensors for distribution throughout the home;
collecting data from the activity sensors indicating a wake up time of the resident;
communicating the collected data to a monitoring center remote from the home via a communication platform; and
analyzing the data collected from the activity sensors to determine if the data indicates the wake up time deviates from a predetermined normal wake up time, signaling an abnormal wake up time.

46. The method of claim 45, wherein if the data indicates the wake up time is consistent with the predetermined normal wake up time, further comprising reporting in near real-time to the caregiver a normal wake up time.

47. The method of claim 45, wherein if the data indicates the wake up time deviates from the predetermined normal wake up time, further comprising contacting the caregiver of the abnormal wake up time.
